(19) United States
(12) Patent Application Publication
(10) Pub. No.: US 2011/0104102 A1
(43) Pub. Date: May 5, 2011

(54) METHOD FOR STIMULATING MAMMALIAN CELLS AND MAMMALIAN CELL

(75) Inventors: Jeri KOISTINAHO, Paukarlahti (FI); Milla KOISTINAHO, Paukarlahti (FI)

(73) Assignee: MEDEIA THERAPEUTICS LTD, Kuopio (FI)

(21) Appl. No.: 12/986,580

(22) Filed: Jan. 7, 2011

Related U.S. Application Data
(62) Division of application No. 11/631,323, filed on Jan. 3, 2007.

(30) Foreign Application Priority Data
Jul. 8, 2004 (FI) 20040953

(51) Int. Cl. A61K 38/19; A61K 35/32; A61P 25/28; A61P 25/16; A61P 25/00; A61P 3/10

(52) U.S. Cl. 424/85.1; 424/93.7

(57) ABSTRACT

The current invention relates to methods for stimulating mammalian cells to enhance their ability to cross the blood-brain-barrier and to phagocytose and degrade beta-amyloid plaques in the brain. The current invention also relates to cells obtained by the method of the invention. The current invention also relates to methods for prevention and treatment of amyloid-accumulating disorders.

### FIELD OF THE INVENTION

The present invention relates to methods for preparing stimulated mammalian cells originated from bone marrow, umbilical cord, any source of hematopoietic stem cells or any other monocytic lineage to enhance their ability to phagocytose and degrade beta-amyloid plaques and beta-amyloid peptides in the brain and to cross the blood-brain barrier. The current invention also relates to cells obtained by said method. The current invention also relates to methods and substances useful for prevention and treatment of disorders or diseases associated with amyloid accumulation, such as Alzheimer's disease or the like.

### BACKGROUND OF THE INVENTION

Disorders associated with amyloid accumulation result when misfolded proteins amass to form amyloids, which are plaque-like deposits that crowd in different organs of the body. These diseases include for example Alzheimer's disease, Parkinson's disease, Huntington's disease, amyotrophic lateral sclerosis (ALS), transmissible spongiform encephalopathies (such as Creutzfeldt-Jacob disease and mad cow disease), type II diabetes, and familial and secondary amyloid diseases (amyloidosis). In these diseases, amyloid fibrils or their soluble precursors are toxic, and experimental evidence indicates that prevention and/or removal of amyloid or its precursors from the diseased tissues is therapeutic. In all these diseases amyloid oligomers are clumps comprising three to 200 of these proteins and are used as building blocks for long fibrous amyloid (See Kayed R. et al., Science 300:486-9 (2003); Klein W L et al., Trends Neurosci. 24:219-24 (2001)).

Transmissible spongiform encephalopathies (TSE) are fatal neurodegenerative diseases that include human disorders such as Creutzfeldt-Jacob disease, kuru, fatal familial insomnia, and Gerstman-Staussler-Scheinker (GSS) syndrome. Animal forms of TSE include scrapie in sheep, chronic wasting disease in deer and elk, and bovine spongiform encephalopathy in cattle. These diseases are characterized by the formation and accumulation of an abnormal proteinase K resistant isoform (PrP-res) of a normal protease-sensitive host-encoded prion protein (PrP-sen) in the brain. PrP-res is formed from PrP-sen by a post-translational process involving conformational changes that convert the PrP-sen into a PrP-res molecular aggregate having a higher beta-sheet content. The formation of these macromolecular aggregates of PrP-res is closely associated with TSE-mediated brain pathology in which amyloid of PrP-res is formed in the brain, which eventually becomes “spongiform”. The presence of both PrP-sen and PrP-res is essential to pathogenesis of TSE. Currently TSEs are incurable. The use of a nerve growth blocking peptide or Congo Red, an amyloid stain, inhibits PrP-res formation and replication of scrapie agent, but has little therapeutic value when infection has reached the central nervous system. Together with inherent toxicity, the utility of these and other potential drugs is very limited.

In type II diabetes the amyloid in the pancreatic islets has amylin (islet amyloid polypeptide, IAPP) as a unique component, but contains other proteins, such as apolipoprotein E and the heparin sulfate proteoglycan perlecan, which are typically observed in other forms of generalized and localized amyloid. Islet amyloid is observed at pathological examination in the vast majority of individuals with type II diabetes but is rarely observed in humans without disturbances of glucose metabolism. Human IAPP can form amyloid fibrils in vitro, but all human subjects produce and secrete the amyloidogenic form of IAPP, yet not all develop amyloid. An alteration in beta-cell function resulting in a change in the production processing, and/or secretion of IAPP is involved in the initial formation of islet amyloid fibrils in human diabetes. This formation of amyloid fibrils may then allow the progressive accumulation of IAPP containing fibrils. The eventual replacement of beta-cell mass by amyloid contributes to the development of hyperglycemia. Currently, individuals with type II diabetes are treated with diet or exercise therapy, or by insulin or insulin release enhancing drugs to reduce the symptoms and complications. There is no known therapy to remove the harmful amyloid or cure the damaged pancreatic tissue.

Amyloidosis can be hereditary or secondary. The symptoms of both types of amyloidosis are the same. Hereditary amyloidoses comprise a clinically and genetically heterogeneous group of autosomal dominant inherited diseases characterized by the deposition of insoluble protein fibrils in the extracellular matrix. These diseases typically present symptoms of polyneuropathy, carpal tunnel syndrome, autonomic insufficiency, cardiomyopathy, and gastrointestinal features, occasionally accompanied by vitreous opacities and renal insufficiency. Other phenotypes are characterized by nephropathy, gastric ulcers, cranial nerve dysfunction, and corneal lattice dystrophy. Rarely, the leptomeningeal or cerebral structures are also involved in the clinical picture. The basic constituents of amyloid fibrils are physiologic proteins that have become amyloidogenic through genetically determined conformation changes. Mutated transthyretin is the most frequent offender in hereditary amyloidosis. Systemic amyloidoses are characterized by the extracellular deposition of fibrillary protein aggregations in parenchymal organs. In addition, any part of the peripheral nervous system may be involved, including nerve trunks and ganglia. Orthotopic liver transplantation may relieve the disease, but with time amyloidosis develops again. Chronic inflammation is a risk factor for secondary amyloidosis. Secondary systemic amyloidosis may occur in association with multiple myeloma, and chronic conditions (those that last for 5 or more years) such as rheumatoid arthritis, tuberculosis, long term paraplegia, bronchiectasis, cystic fibrosis, chronic osteomyelitis, recurrent pyogenic (involving pus) skin infection/abscess, decubitus ulcers, chronic renal dialysis, juvenile chronic arthritis, systemic lupus erythematosus, Reiter's syndrome, ankylosing spondylitis, Hodgin's disease, Sjogren's syndrome, and hairy cell leukemia.

Parkinson's disease is a progressive neurological disorder marked by tremors, muscle rigidity, and balance and coordination problems. The destruction of neurons that produce the chemical dopamine underlies these symptoms. The degenerating dopamine-producing neurons are also associated with protein deposits called Lewy bodies. The mechanisms and the role of Lewy bodies in neurodegeneration are not known. However, mutations in the genes encoding two proteins, called parkin and alpha-synuclein, are linked to separate, rare forms of inherited Parkinson's disease, and are found in Lewy bodies that build up in the brains of all Parkinson's disease patients. Moreover, recent findings suggest that parkin plays an important role in regulating proteins associated with Lewy bodies, including alpha-synuclein and synphilin. Normally, parkin uses another protein, called ubiquitin, to tag other proteins for destruction. When the interaction between these proteins is disturbed, the process leading to cell death in Parkinson's disease may occur. Both parkin and alpha-synuclein are linked with synphilin-1 in a common pathogenic mechanism involving the ubiquitination of Lewy body-associated proteins. Alpha-synuclein may be the core of both the inherited and common forms of Parkinson's disease. Generally patients with Parkinson's disease are treated with drugs which either increase dopamine concentrations or reduce acetylcholine concentrations in the brain, but these drugs loose their effect with time, and have no impact on disease progression.

Alzheimer's disease (AD) is an amyloid disorder that destroys cells in the brain. The disease is the leading cause of dementia, a condition that involves gradual memory loss, decline in the ability to perform routine tasks, disorientation, difficulty in learning, loss of language skills, impairment of judgment, and personality changes. As the disease progresses, people with Alzheimer's disease become unable to care for themselves. The loss of brain cells eventually leads to the failure of other systems in the body. The rate of progression of Alzheimer's disease varies from person to person. The time from the onset of symptoms until death ranges from 3 to 20 years. The average duration is about 8 years. The diagnosis is confirmed histologically by the presence of beta-amyloid containing plaques and neurofibrillary tangles in the brain. Beta-amyloid, derived from beta-amyloid precursor protein (APP), plays a central etiological role in the disease. In a healthy brain, these soluble protein fragments would be broken down and eliminated. In Alzheimer's disease, the fragments accumulate to form oligomers and eventually hard, insoluble plaques. The histopathological features observed in the different forms of AD are strikingly similar, although the disease is ethiologically heterogeneous. A small portion of AD cases is caused by autosomal dominant mutations in APP or presenilin genes that add to elevation of highly fibrillogenic form of beta-amyloid. The disease can occur as a sporadic event or it can result from triplication of APP gene-containing chromosome 21 (Down's syndrome). Additional genetic complexity is caused by the fact that the epsilon 4 allele of apolipoprotein E is the main risk factor for late-onset, sporadic form of AD. The mechanism on how APP increases neuronal vulnerability in AD is not completely clear, but beta-amyloid, which constitutes a collection of peptides of 39-43 residues in length, can assume a number of oligomeric and aggregated forms. Beta-amyloid is toxic to neurons both in the aggregated and soluble forms. The current main treatment protocols include two classes of drugs to treat memory symptoms of Alzheimer's disease. The first specific Alzheimer medications to be approved were cholinesterase inhibitors. They work by temporarily increasing the brain's supply of acetylcholine, a cell-to-cell communication chemical involved in learning and memory that becomes deficient in the Alzheimer brain. Another drug memantine works by regulating the activity of glutamate, another cell-to-cell communication chemical. Some glutamate is needed for learning and memory, but too much can overstimulate and damage nerve cells. Memantine protects brain cells against the effects of excess glutamate. However, neither cholinesterase inhibitors nor memantine are able to slow down or stop the progression of the disease and therefore they offer only temporary ease for some patients. Physicians also often prescribe vitamin E because it may reduce molecular activity contributing to brain cell damage. However, vitamin E has not been shown to have any significant effect on disease progression or symptoms. Other medications may be prescribed to treat such symptoms as agitation, anxiety, depression, and poor sleep. Several new strategies for treating Alzheimer's disease have been proposed and they include decreasing or preventing the release of beta-amyloid peptide by either increasing alpha-secretase or decreasing beta or gamma-secretase activity or production. Other strategies include immunological control of beta-amyloid levels. However, this approach has been shown to have severe side effects, such as brain hemorrhages and encephalopathy.

Amyotrophic lateral sclerosis (ALS), sometimes called Lou Gehrig's disease, is a rapidly progressive, invariably fatal neurological disease that attacks the neurons responsible for controlling voluntary muscles. The disease belongs to a group of disorders known as motor neuron diseases, which are characterized by the gradual degeneration and death of motor neurons. Motor neurons are nerve cells located in the brain, brainstem, and spinal cord that serve as controlling units and vital communication links between the nervous system and the voluntary muscles of the body. Messages from motor neurons in the brain (called upper motor neurons) are transmitted to motor neurons in the spinal cord (called lower motor neurons) and from them to particular muscles. In ALS, both the upper motor neurons and the lower motor neurons degenerate or die, ceasing to send messages to muscles. Unable to function, the muscles gradually weaken, waste away (atrophy), and twitch (fasciculations). Eventually, the ability of the brain to start and control voluntary movement is lost. ALS causes weakness with a wide range of disabilities. Eventually, all muscles under voluntary control are affected, and patients lose their strength and the ability to move their arms, legs and body. When muscles in the diaphragm and chest wall fail, patients lose the ability to breathe without ventilatory support. Most people with ALS die from respiratory failure, usually within 3 to 5 years from the onset of ALS symptoms. The cause of ALS is not known, but mutations in the gene that produces the SOD1 enzyme are associated with some cases of familial ALS. This enzyme is a powerful antioxidant that protects the body from damage caused by free radicals. Free radicals are highly unstable molecules produced by cells during normal metabolism. If not neutralized, free radicals can accumulate and cause random damage to the DNA and proteins within cells. Although it is not yet clear how the SOD1 gene mutation leads to motor neuron degeneration, researchers have theorized that protein aggregates containing SOD1 accumulate free radicals, which may result from the faulty functioning of this gene. The toxicity of SOD1 containing aggregates may also involve glutamate, which is one of the chemical messengers or neurotransmitters in the brain. Compared to healthy people, ALS patients have higher levels of glutamate in the serum and spinal fluid. Laboratory studies have demonstrated that neurons begin to die off when they are exposed over long periods to excessive amounts of glutamate. No cure has yet been found for ALS. Riluzole, which is believed to reduce damage to motor neurons by decreasing the release of glutamate, may prolong survival by months, mainly in those with difficulty swallowing. Riluzole does not reverse the damage already done to motor neurons, and patients taking the drug must be monitored for liver damage and other possible side effects. Other treatments for ALS are designed to relieve symptoms and improve the quality of life for patients.

Huntington's disease is a progressive, autosomal, dominantly inherited, neurodegenerative disease that is characterized by involuntary movements (chorea), cognitive decline and psychiatric manifestations. This is one of a number of late-onset neurodegenerative disorders caused by expanded glutamine repeats, with a likely similar biochemical basis. Immunohistochemical studies have identified neuronal inclusions within densely stained neuronal nuclei, peri-nuclear and within dystrophic neuritic processes. However, the functional significance of inclusions is unknown. It has been suggested that the disease-causing mechanism in Huntington's disease (and the other polyglutamine disorders) is the ability of polyglutamine to undergo a conformational change that can lead to the formation of very stable anti-parallel beta-sheets; more specifically, amyloid structures. Some inclusions in Huntington's disease brain tissue possess an amyloid-like structure, suggesting parallels with other amyloid-associated diseases such as Alzheimer's and prion diseases. There is no cure for Huntington's disease, and there is no known way to stop progression of the disorder. Treatment is aimed at slowing progression and maximizing ability to function for as long as possible. Medications vary depending on the symptoms. Dopamine blockers such as haloperidol or phenothiazine medications may reduce abnormal behaviors and movements. Reserpine and other medications have been used, with varying success. Drugs like Tetrabenazine and Amantadine are used in trials to control extra movements. There has been some evidence to suggest that co-enzyme Q10 may minimally decrease progression of the disease. Symptomatic treatment for the dementia is similar to that used for any organic brain syndrome.

One common feature for all amyloid-related diseases is inflammation, which in the brain is manifested as activation and proliferation of microglia cells. Microglia are phagocytic cells of the brain. (See Neuroscience. 2nd ed. Purves, Dale; Augustine, George. J.; Fitzpatrick, David; Katz, Lawrence. C.; LaMantia, Anthony-Samuel.; McNamara, James. O.; Williams, S. Mark, editors. Sunderland (Mass.), Chapter 1: Sinauer Associates, Inc. 2001; Cuadros and Navascues, Prog Neurobiol 56:173-189 (1998)). Microglia constitute a small percentage of all non-neuronal cells in the brain and they are generally in a ramified, resting state in the normal brain. When activated, they acquire ameboid morphology and produce a variety of receptors and other molecules involved in inflammation and phagocytosis. Activated microglia are associated with amyloid in amyloid-related brain diseases, and the overall number and activity of microglia is strongly increased in degenerating brain areas. The relationship of microglia and amyloid or neuronal death in development of amyloid-related disease is still unclear. Certain compounds, which inhibit microglial activation, have been reported to slow down the disease progression in animal models of brain amyloid diseases. In addition, the use of certain antiinflammatories, such as ibuprofen, which also inhibits microglial activation, reduces the incidence of certain types of Alzheimer's disease. On the other hand, in animal models of Alzheimer's disease, antibodies to beta-amyloid and overexpression of tumor growth factor-beta gene activate microglia around amyloid plaques, resulting in beta-amyloid phagocytosis and clearance (Janus, CNS Drugs 17:457-474 (2003)). Moreover, stimulation of microglia with macrophage colony factor or lipopolysaccharide induce phagocytic properties in vitro, and lipopolysaccharide-induced activation of microglia occurs parallel to clearance of beta-amyloid. However, no evidence has been provided that microglia would be responsible for beta-amyloid clearance after lipopolysaccharide stimulation in vivo (Mitrasinovic O. M. et al., Neurosci Lett. 344:185-188 (2003); DiCarlo G. et al., Neurobiol. Aging 22:1007-1012 (2001); Abd-Basset & Federoff, J. Neurosci. Res. 41:222-237 (1995)). Mentlein, R. et al. Journal of Neurochemistry, 70:721-726 (1998). McGeer, P. et al. (Science of Aging Knowledge Environment 27. pe29 (2004)) disclose that vaccination with beta-amyloid leads to activation of microglial phagocytosis of beta-amyloid.

Recently it has been shown that bone marrow derived cells migrate into the brain of APP-PS1 double transgenic mice of Alzheimer's disease (T. Lappeteläinen, M. Koistinaho, T. Vatanen, A. Ooka, S. Karlsson, J. E. Koistinaho. BONE MARROW DERIVED CELLS MIGRATE INTO THE BRAIN OF APP-PS1 DOUBLE TRANSGENIC MICE OF ALZHEIMER'S DISEASE. Program No. 945.4. 2003 Abstract Viewer/Itinerary Planner. Washington, D.C.: Society for Neuroscience, 2003. Online). To investigate whether some of beta-amyloid-associated cells are blood-derived cells recruited during disease development, 21 month-old APP-PS1 double transgenic mice (n=4) and their wild type controls (n=4) were lethally irradiated (550 cGy twice, 3 hours apart) and the next day transplanted with bone marrow isolated from 6-8 week old eGFP over expressing mice. The ability of the bone marrow derived cells to engraft the bone marrow (BM) and to produce new blood cells was analyzed 4 weeks after the transplantation using flow cytometry. The number of eGFP-positive cells in different brain regions and their distribution relative to the beta-amyloid plaques was analyzed 14 weeks after the transplantation.

All the mice survived through the BM cell transplantation. Flow cytometry analysis showed that 75% of the MAC-1 positive blood cells were eGFP fluorescent. The number of the BM cells infiltrated into the brain was the same in APP-PSI trans-genic and control mice. In APP-PSI mice, some eGFP positive cells were associated with beta-amyloid plaques, suggesting that BM-derived cells continuously infiltrate into the brain contributing to beta-amyloid plaque pathology in Alzheimer's disease. However, the transplanted eGFP-positive BM-derived cells did not show increased migration through the blood brain barrier. Neither were the eGFP-positive BM-cells associated with beta-amyloid plaques shown to be able to phagocytose or degrade beta-amyloid plaques.

WO 0204604 A2 describes a genetically modified immortalized human microglia cell line which has the characteristics of a human embryonic microglia. This cell line has demonstrable phagocytic properties in vitro and it contains human genomic DNA which has been genetically modified to include a viral vector carrying at least one DNA segment encoding an exogenous gene for intracellular expression. WO 0204604 A2 speculates of microglia cells capable of bypassing the blood-brain-barrier to deliver drugs into brain, but does not provide any data for implementing that. The phagocytic capacity of said human cell line has been demonstrated by exposing the cells for latex beads or for carbon particles in vitro whereupon the cells became loaded with latex beads or carbon particles. No phagocytic capacity on amyloid plaques or the like has been demonstrated. Instead, WO 0204604 A2 demonstrates release of toxic molecules upon stimulation with inflammatory agents or amyloid, implicating that these cells may induce neuronal damage in the brain. Importantly, previous investigations have demonstrated that human microglia, which were also used to support the claims in WO 0204604 A2, are not particularly able to degrade amyloid (Bard et al., Nat Med. 6:916-919 (2002) and Rogers et al., Glia. 40:260-269 (2002)).

EP0949331 discloses an established cell line of microglia from mice, which has a specific affinity for the brain and phagocytic ability, but the cell line was shown to release neurotoxic molecules, such as IL-1 and IL-16. Moreover, the cell line described is derived from the brain tissue. Whether this cell line truly has specific affinity for the brain is unclear since migration of these cells to the brain was compared only to the liver. In addition, the latest time point investigated after injection was 3 weeks, indicating that the difference in tissue infiltration between the liver and brain may be lost after longer follow-up times. Moreover, even though the cell line established in EP0949331 would have a phagocytic capacity in general, it does not indicate that these cells would be able to phagocytose and degrade amyloid in vivo.

However, the use of human embryonic cells for development of therapy has not been legally or even ethically approved in the USA or numerous other Western countries, and utilization of cells from human newborns has similar ethical problems. Also, brain tissue as a source of the cells to be utilized for the treatment is both ethically and technically very challenging and complicated. Therefore, there is a need for a cell deriving from adult individuals, particularly from adult (peripheral) tissues, which are accessible easily and without risks, and for a cell with ability to phagocytose amyloids, such as beta-amyloid. Preferably said cell should be able to cross the blood-brain-barrier to be able to act in the brain. The use of microglial cells should be avoided because microglia can phagocytose brain-derived amyloid only after stimulation with beta-amyloid antibodies. Further, microglia needs to be purified from the brain which limits the cell number available for transplantation and includes a risk of impurities (such as material of other cell populations). Moreover, activated microglia has been reported to release very potent neurotoxins.

### SUMMARY OF THE INVENTION

It is an object of the present invention to provide new methods and substances useful for treating disorders where amyloid accumulates in the tissue. The present invention provides methods for transforming mammalian cell originated from bone marrow, umbilical cord, any source of hematopoietic stem cells or other monocytic lineage into a phagocytosis-stimulated form, which can degrade beta-amyloid or constituents thereof, such as beta-amyloid plaques, in a tissue. Preferably said cells are of non-embryonic origin. Further, the stimulated cells are able to cross the blood-brain barrier and degrade said beta-amyloid or constituents thereof in tissue, such as brain tissue or nerve tissue. These cells may further be used for prevention and/or treatment of disorders, for example they can be injected to patients suffering from disorders or diseases in which amyloid accumulates in tissues, and degradation or removal thereof is desired, such as in Alzheimer's disease or the like.

The stimulation of the cell is accomplished by treating it with an agent, which will cause a phagocytotic response on said cell to transform said cell into a phagocytotic form, which is capable of degrading beta-amyloid or constituents thereof in the tissue. Said response is relatively quick, starting within 3-7 days after stimulating the cells, and it is naturally enhanced by beta-amyloid pathology, which increases the migration of bone marrow cells into the diseased tissue, such as the brain regions, which contain beta-amyloid. In addition, the stimulated cells do not damage neurons in the brain.

An advantage of the invention is that a new efficient and specific method for preventing and treating amyloid accumulating disorders is provided compared to the current treatment protocols, which do not alter the levels of amyloid or trigger degradation of beta-amyloid to protect the brain against toxic amyloid protein. By reducing beta-amyloid levels in the brain, the present invention slows down major tissue pathology, which is the progression of amyloid accumulation. The treatment described in the present invention is cheap and relatively easily carried out.

A further advantage of the invention is that the migration of the cells of the present invention has high relative tissue selectivity, as the stimulatory treatment can be given inside the central nervous system by administration to the cerebrospinal fluid. This tissue selectivity prevents the potential side effects that would be caused by systemic stimulation of bone marrow cells or the like.

A further advantage of the invention is that the cells of the present invention can have either allogenic or autogenic origin, making it possible to use individual's own cells without the risk of rejection.

Another advantage is that the transplanted cells of the present invention have distinct properties, making them different from endogenous brain microglia, which do not respond by phagocytic activity as efficiently as the cells of the present invention, but may release, instead, neurotoxic molecules upon immunological stimulation.

Still another advantage is that the cell of the present invention may originate from non-embryonic tissue, such as bone marrow, umbilical cord or hematopoietic stem cells, and it is therefore accessible without ethical or technical difficulties.

Still another advantage compared to brain-derived cells, such as microglia, is that the cells of the present invention are available in high amounts sufficient to carry out transplantations.

One aspect of the present invention relates to a method for preparing a stimulated bone marrow cell, umbilical cord cell, hematopoietic stem cell or a cell from other monocytic lineage, which is capable of degrading amyloid or constituents thereof in a tissue.

Another aspect of the present invention relates to stimulated cell obtained by said method.

Still another aspect of the present invention relates to said stimulated cell of the present invention for use as medicament for treating amyloid-accumulating disorders, such as Alzheimer's disease, Parkinson's disease, Huntington's disease, amyotrophic lateral sclerosis (ALS), transmissible spongiform encephalopathy (such as Creutzfeldt-Jacob disease and mad cow disease), type II diabetes and familial and secondary amyloid diseases (amyloidosis).

Still another aspect of the present invention relates to the use of said cell of the present invention for treating amyloid-accumulating disorders.

Still another aspect of the present invention relates to a method for treating amyloid-accumulating disorders.

The present invention is now explained in detail by referring to the attached figures and examples. These examples are only used to show some of the embodiments and are not intended to limit the scope of the invention.

### BRIEF DESCRIPTION OF THE DRAWINGS

The invention is described in detail below. The description refers to the enclosed drawings, in which

FIG. 1 shows a graph (A) illustrating how migration of transplanted bone marrow cells in the brain is increased in the transgenic (TG) mouse model of AD when compared with healthy wild-type (WT) mouse. Several green fluorescent (arrows) BM-derived cells were associated with Aβ (red fluorescence) deposits (B). Scale bar=50 μM.

FIG. 2 shows migration, activation and phagocytotic activity of BM cells after LPS, M-CSF or SDF-1 a treatment. When AD mice have received BM transplantation coupled with LPS, M-CSF or SDF-1α treatment, the migration of transplanted bone marrow cells into the brain is increased about 10-fold (A, fluorescence area=brain area covered by fluorescent BM cells), resulting in degradation (clearance) of beta-amyloid (Aβ) (B, immunoreactive area=brain area covered by Aβ immunoreactive material) in the brain of transgenic mouse model of AD. The density of MHCII-immunoreactive BM cells increased substantially after LPS or SDF-1α treatment (C, immunoreactive area=brain area showing immunoreactivity for MHCII). Also, the number of BM-derived cells associated with Aβ deposits was increased after LPS treatment (D). The increased phagocytotic activity of LPS, M-CSF or SDF-1α treated BM cells (E).

FIG. 3 shows confocal microscopy demonstration that Aβ-immunoactivity is colocalized within eGFP-positive BM cell after LPS treatment, indicating induction of phagocytic activity of these cells.

### DETAILED DESCRIPTION OF THE INVENTION

The mammalian cell of the present invention to be stimulated can be obtained from any suitable mammalian species, such as human, rodent or other species, by any suitable isolation method. The cells may originate from any suitable source, such as bone marrow, umbilical cord or any source of hematopoietic stem cells or other monocytic lineage. Generally said bone marrow cells are hematopoietic stem cells. Microglial cells or other fully differentiated bone marrow derived cells found in tissues are not in the scope of the present invention. In the method of the invention the cell can be stimulated in any suitable way. One example is a treatment of the cell by contacting it with a chemical or a biochemical agent which triggers the transformation of said cell into a phagocytotic form. This activated cell can degrade amyloid or constituents thereof and it is able to cross the blood-brain-barrier.

The present invention provides a method for preparing a stimulated mammalian cell comprising stimulating said cell by treating it with a phagocytosis-stimulating agent to cause a response on said cell to transform said cell into a phagocytotic form capable of degrading beta-amyloid or constituents thereof in a tissue.

The present invention also provides a stimulated mammalian cell, which has been stimulated by treating it with phagocytosis-stimulating agent to cause a response on said cell to transform said cell into a phagocytotic form capable of degrading beta-amyloid or constituents thereof in a tissue.

As used herein, the “phagocytotic stimulation of a cell” refers to any treatment, such as chemical or biochemical treatment, which is given to said cell in vitro or in vivo, acts directly on or inside said cell, and results in immunological phagocytotic activation of this particular cell towards beta-amyloid-containing deposits and the like, as described in the specification.

In one embodiment said cell is substantially undifferentiated. “Substantially undifferentiated” cell as used herein refers to a cell not fully differentiated into tissue macrophages, Kuppfer cells, microglial cells or the like. Non-limiting examples of such cells are bone marrow cells as defined above, umbilical cord cells, any source of hematopoietic stem cells or cells from any other monocytic lineage. For example microglial cells are excluded.

In one embodiment said cell is a bone marrow cell or a bone marrow derived cell. As used herein, “bone marrow cell” or “bone marrow derived cell” refers to bone marrow cells, bone marrow-derived cells or other hematopoietic stem cells (HSC) of mammalian origin and which cells are derived from the red marrow or any other suitable tissue of the body and which are developmentally of same cellular origin. Preferably said cells are substantially undifferentiated. Even though the fully differentiated microglial cells, macrophages, Kuppfer cells and alike are derived from myeloid progenitor cells which originate from the bone marrow, they are excluded from the definition of “bone marrow derived cells”, because these particular cells of monocytic lineage are fully differentiated and do not enter the tissues as well as undifferentiated bone marrow derived cells do. Another reason for excluding these differentiated cells, such as microglia, is that they do not degrade beta-amyloid deposits (in the form they are present in tissues) ex vivo or in vivo unless these deposits have been opsonized with specific and certain beta-amyloid antibodies. Moreover, transplanted bone marrow cells maintain the immunological and morphological characteristics different from fully differentiated microglial cells even several months after the transplantation.

In another embodiment said cell is an umbilical cord cell. In still another embodiment said cell is a hematopoietic stem cell or any origin of monocytic lineage. Because of the developmentally same origin, the same cellular and functional properties, and similar morphological phenotypes, the umbilical cord cells and hematopoietic cells of any source are considered equal to bone marrow cells used in most of the examples in the specification. All said cells have substantially the same effect and behavior in the present invention, i.e. what is said herein on bone marrow cells is also comparable with umbilical cord cells or hematopoietic cells.

In one embodiment the stimulation of the cells is done in vitro. In another embodiment said cells to be stimulated are administered to the patient and any of said stimulating agents is administered separately or together with the cells.

In one embodiment said phagocytosis-stimulating agent comprises lipopolysaccharide (LPS). As used herein, “lipopolysaccharide” refers to the major constituent of the cell walls of gram-negative bacteria or to endotoxin of any other bacteria. Lipopolysaccharides can be for example isolated and purified to any suitable purity from bacteria or alike, or be synthesized and eventually administered at any suitable concentration and purity.

In another embodiment said phagocytosis-stimulating agent comprises macrophage colony stimulating factor (M-CSF). As used herein, “macrophage colony stimulating factor” refers to a 70 kD glycoprotein dimer, which is a growth factor that causes the committed hematopoietic cell to proliferate and mature into phagocytous cells of monocyte-macrophage series upon binding to a single class of high-affinity receptor. M-CSF's may originate from any mammalian or other species and be isolated or synthesized to suitable purity.

In still another embodiment said phagocytosis-stimulating agent comprises stromal cell derived factor-1 alpha (SDF-1). As used herein “stromal cell derived factor-1 alpha” refers to 7.9 kDa protein, a CXC chemokine, which binds to chemokine receptor CXCR4. SDF-1α is a chemoattractant of bone marrow derived cells. SDF-1α may originate from any mammalian or other species and be isolated or synthesized to suitable purity. In still another embodiment a combination of two or more of said phagocytosis-stimulating agents is used.

As used herein, the term “mammalian” refers to any individual of mammalian species, including rodents (gerbils, rats, mice and the like), large animals (cows, sheep, horses and the like), sport animals (including dogs and cats), and primates (including old world monkeys, new world monkeys, apes, humans, and the like). In another embodiment said mammalian is human, mouse or rat.

In one embodiment said tissue is brain tissue or nerve tissue.

The present invention also provides said stimulated cell for use as medicament. Furthermore, the present invention also provides said medicament or pharmaceutical composition, the use of said cell as well as a method for treating or preventing amyloid-accumulating disorders wherein degradation of amyloid or constituents thereof in a tissue is desired. Generally a pharmaceutically effective amount of said stimulated cells can be administered to a patient in need thereof. In one embodiment the cell is adapted to be administered to the cerebrospinal fluid.

As used herein, “amyloid-accumulating disorder” refers to any human or other mammalian disorder or disease, which is known to accumulate stable, highly organized protein aggregates known as amyloid fibrils in any tissue. Some amyloid-accumulating diseases are described by a way of example in the specification, which however should not be considered as limiting the scope of the invention. To prevent or treat such a disorder it is desired to degrade said aggregates in the tissue.

The cells according to the invention can be administered to the patient by any suitable means known in the art, such as injection or infusion. The injection can be given to cerebrospinal fluid, on top of the dura mater, subcutaenously, intradermally, intraperitoneally, intravenously, intra-arterially or into any tissue, such as brain tissue. The patient to be treated can be any mammalian, as defined above, preferably human, who suffers from an amyloid-accumulating disorder, such as Alzheimer's disease, Parkinson's disease, Huntington's disease, amyotrophic lateral sclerosis (ALS), transmissible spongiform encephalopathy (such as Creutzfeldt-Jacob disease or mad cow disease), type II diabetes or familial or secondary amyloid disease (amyloidosis). The amount of cells to be administered can be determined easily by a person skilled in the art, such as a physician. Any suitable pharmaceutically acceptable carriers or compositions may be administered together with the cell of the invention.

In the following examples three different agents, lipopolysaccharide (LPS), macrophage colony stimulating factor (M-CSF) and stromal cell-derived factor-1 alpha (SDF-1α) have been used as phagocytosis-stimulating substances for bone marrow cells. With all three agents a remarkable and similar stimulation of bone marrow cells could be detected when compared to healthy control mouse. The in vitro experiments showed that LPS, M-CSF and SDF-1α enhance beta-amyloid degradation by bone marrow cells. Also an in vivo experiment has been carried out to show how tissue-specific injection of stimulating agent will increase the amount of bone marrow cells in said tissue. Experiments not included herein also show that the same effect is achieved with umbilical cord cells or hematopoietic cells by using the same stimulating substances.

In the experiments carried out with transgenic AD mice expressing chimeric mouse/human vectors it is also shown that bone marrow cells are able to cross the blood-brain-barrier. Also, the beta-amyloid deposits attract said cells to close proximity of the deposits.

The following examples and results are provided to demonstrate the present invention only in an illustrative way and they should not be considered as limiting the scope of the invention. The amounts of stimulating agents and cells to be used are only exemplary and may depend on the cells, agents or patients to be treated. A person skilled in the art may define proper amounts or dosages with methods well known in the art.

### EXAMPLES

### Example 1

### Transplantation of BM Cells into Transgenic Alzheimer Mouse and Increased Engraftment of Transplanted BM Cells into Alzheimer Mouse Brains

### Transgenic Mice

The mice used were generated by co-injection of chimeric mouse/human APPswe and human PS1-dE9 vectors, both controlled by their own mouse prion protein promoter element (Jankowsky et al., Hum Mol Genet. 13:159-170 (2004)). The double transgenic mice (APPdE9) were backcrossed to C57BL/6J strain for six generations. Altogether 5 2.5-month-old female APPdE9 transgenic and 5 age-matched wild-type controls were used in this study. Enhanced GFP (eGFP) overexpressing mice (Okabe et al., FEBS Lett. 407:313-319 (1997)), were purchased from Jackson Laboratories (Maine, USA) and were maintained in C57BL/6J strain in the Animal Facilities of the National Public Health Institute in Kuopio, Finland. All animal experiments were carried out according to the National Institute of Health (NIH) guidelines for the care and use of laboratory animals, and approved by the Ethical Committee of the National Laboratory Animal Center, University of Kuopio, Finland.

### Bone Marrow Transplantation

APP+PS1 and APPdE9 double transgenic mice and their age-matched wild-type controls were lethally irradiated with two doses of 550 cGy 3 hours apart with the dose rate 2.37 Gy/min (Varian 600 C Radiotherapy Accelerator, 4 MV high-energy x-rays). 1-cm-thick custom-made polymethylmetacrylate lucite beam spoiler (scatterer) was used to ensure sufficient surface dose. The irradiated mice were transplanted the next day with BM cells (5×10⁶ cells) by tail vein injection (Priller et al., Nat Med. 7:1356-1361 (2001)). BM cells were isolated from 6 to 8 week-old donor eGFP overexpressing mice by flushing the femur and tibias with Hank's balanced salt solution (Bio Whittaker Europe, Belgium) containing 10% fetal bovine serum (FBS, Gibco, BRL/LifeSciences) in a protocol similar to the one described earlier (Kennedy and Abkowitz, Blood 90:986-993 (1997)). All the transplanted mice survived throughout the follow-up period, indicating successful engraftment of BM cells.

Flow Cytometry Analysis of eGFP Expression in Peripheral Blood Cells

The ability of BM cells to engraft the BM and produce new blood cells was analyzed 8 weeks after the transplantation using a dual laser flow cytometer (Becton Dickinson, Mountain View, Calif., USA). Blood samples were collected and stained with following antibodies: PE-conjugated Ly-6G to detect granulocytes, PE-conjugated CD11b to detect monocytes, PerCP-conjugated CD3e to detect T-cells and PerCP-conjugated CD45R/B220 to detect B-cells (all from BD Biosciences, NJ, USA). Briefly, blood samples were collected from femoral vein into heparinized Eppendorf tubes. 50 μl of blood was incubated with antibodies mentioned above in the presence of a blocking antibody mouse IgG1 (Sigma; 10 μg/ml) on ice for 30 minutes. The cells were centrifuged and lysed with 150 mM NHCl₄, 10 mM KHCO₃, 0.1 mM EDTA pH 7.4. After washing twice with phosphate-buffered saline (PBS) containing 2% FBS (Gibco, BRL/LifeSciences), the cells were resuspended in PBS/2% FBS and fixed with 2% formalin for FACS analysis. Data were evaluated using the Cellquest™ software (BD immunocytometry systems, CA, USA). It was found that over 90% of the CD11b-positive peripheral monocytes were eGFP-fluorescent, indicating successful replacement of endogenous monocytic cells by transplanted BM cells.

Table 1 shows that the transplanted eGFP positive bone marrow cells were able to engraft the bone marrow of the recipient mice and produce a variety of blood cells as analyzed by flow cytometry. Table shows the percentage of eGFP positive cells from monocytes (CD11b), granulocytes (Ly6G), B-cells (CD45/B220) and T-cells (CD3e) in three transplantation experiments. Values are presented as mean±SEM.

TABLE 1
Engraftment analysis: The percentage of eGFP positive cells from
different blood cell types.

| | CD3e | CD45/B220 | Ly-6G | CD11b |
|---|---|---|---|---|
| Study I | 64.9 ± 4.1 | 91.4 ± 0.7 | 86.1 ± 2.7 | 90.7 ± 1 |
| 2.5-month-old APdE9 | | | | |
| mice | | | | |
| Study II | 74.1 ± 2.3 | 81.3 ± 2.0 | 79.4 ± 1.0 | 87.4 ± 2.4 |
| 25-month-old | | | | |
| APP + PS1 mice | | | | |

Analysis of eGFP Positive Cells and Histology of the Brain

The mice were anesthetized with pentobarbital and flushed transcardially with saline followed by perfusion with 4% paraformaldehyde 26 weeks after the transplantation. The brains were removed and postfixed by immersion in the same fixative for 12 hours at 4° C. After cryoprotection in 30% sucrose for 3 days, the brains were frozen in liquid nitrogen. 10-μm-thick coronal cryosections were used for immunohistochemistry. The number of eGFP positive cells in different brain regions and their distribution relative to Aβ deposits were analyzed immunohistochemically using an antibody against human Aβ (clone 6E10, dilution 1:1000, Signet Laboratories Inc., MA, USA) and visualizing the green fluorescent cells under appropriate filter sets with a fluorescence (Olympus AX70, Olympus, N.Y., USA) or a confocal microscope (BioRad Radiance Laser Scanning Systems 2100, Bio-Rad Microscience Ltd, Hertfordshire, UK) running LaserSharp 2000 software (Bio-Rad Microscience Ltd). For the detection of human Aβ under fluorescence, Alexa Fluor 568-conjugated secondary antibody (Molecular Probes, Eugene, Oreg., USA) was used. For the visualization under light microscope, incubation with biotinylated rabbit anti-mouse IgG (Vector Laboratories Inc., Burlingame, Calif., USA) was followed by avidin-biotin complex (Vectastain Elite kit, Vector Laboratories Inc., Burlingame, Calif., USA) and the immunoreaction was visualized using diaminobenzidine (DAB) or nickel enhanced diaminobenzidine (NiDAB) (Sigma Aldrich Chemie, Germany) as substrates. Counting of eGFP expressing cells was done by an observer blind to the genetic and/or treatment status of the mice based on the visibility of a cell soma in 10-μm-thick coronal sections. Four to six coronal sections at 200 μm intervals at the hippocampal level were evaluated per animal and the cells were counted from the whole section.

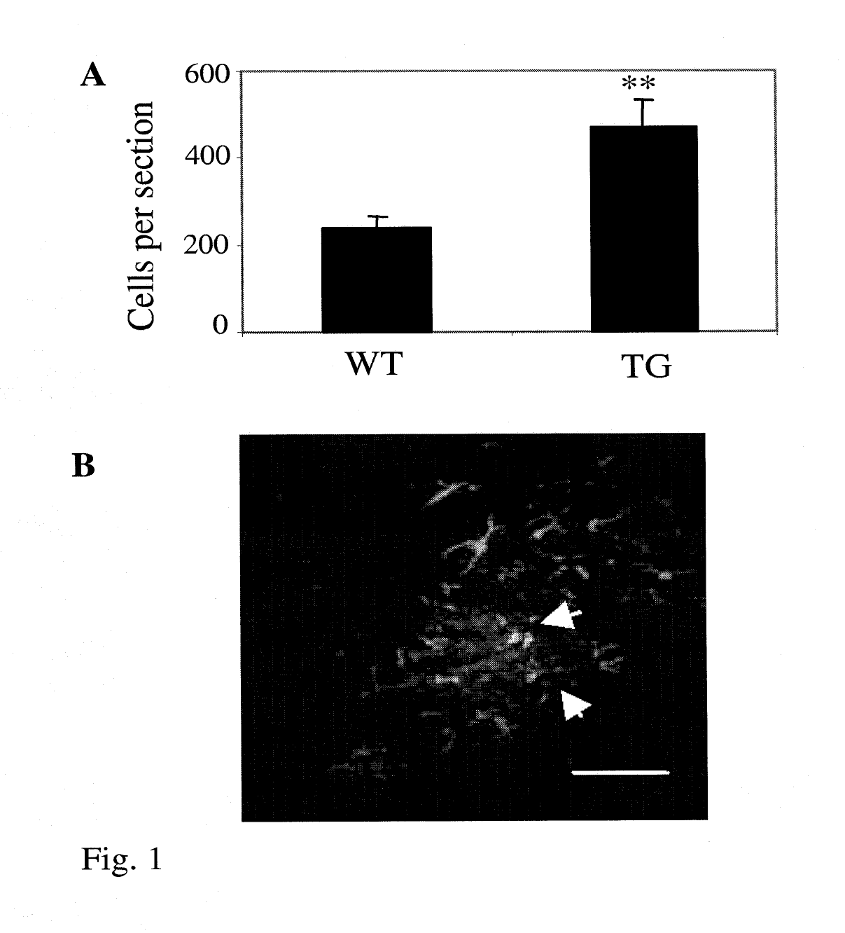

It was found that by the time of sacrifice at the age of 8.5 months, all the mice showed Aβ deposition in the forebrain. The number of eGFP positive cells found in the brains of these transgenic mice was 170% higher compared to the age-matched wild type controls (FIG. 1A) (repeated measures ANOVA, p<0.05). These eGFP positive, BM cells were positively labeled with isolectin B4. Moreover, in these transgenic mice, approximately 10% of the eGFP positive cells were associated with Aβ deposits (FIG. 1B). These results indicate that BM-derived cells cross the blood-brain barrier and their migration to the brain is increased by the presence of beta-amyloid deposits, which attract BM-derived cells to close proximity of the deposits.

### Example 2

### Increased Brain-Specific Engraftment of Transplanted BM Cells into the Brain and Activation of Transplanted BM Cells to Phagocytose and Clear Amyloid

Double transgenic female mice carrying chimeric mouse/human APP695 harboring the Swedish mutation (K595N/M596L) and human PS1 with familial AD-linked A246E mutation (Borchelt et al., Neuron. 19:939-945 (1997)) were used in this experiment. The parental APP695swe and PS1 (A246E) mice were backcrossed 13-14 generations to C57BL/6 strain after which they were intercrossed to create double transgenic mouse line (APP+PS1 mice). Altogether 8 25-month-old APP+PS1 mice and 8 age-matched wild-type controls per age group were used in this study.

Flow cytometry analysis of eGFP expression in peripheral blood cells was done as described in Example 1 and showed that about 90% of the CD11b-immunoreactive monocytes were GFP positive, indicating successful engraftment of BM transplants.

Stimulation of BM-Derived Cells with LPS, M-CSF and SDF-1α In Vivo

APP+PS1 double transgenic mice transplanted with BM-derived cells at the age of 25 months were injected with 0.9% NaCl (saline) into the left hippocampus and 4 μg of LPS (4 μg/μl in saline; Lipopolysaccharide from Salmonella typhimurium, Sigma), 1.0 μg M-CSF (1.0 μg in saline; M-CSF from R&D Systems) or human recombinant SDF-1α (0.1 μg in saline; SDF-1α from R&D Systems) into the right hippocampus according to the following coordinates: M/L±2.5 mm, NP −2.7 mm, D/V −3 mm 16 weeks after the transplantation. The mice were anesthetized with halothane and placed in a stereotaxic apparatus (David Kopf, model 940, Tujunga, Calif., USA). Two holes were drilled in the cranium above the hippocampi and injections were made using a 5 μl syringe (Hamilton, Reno, Nev.) over a period of 10 minutes. The incision was cleaned with saline and closed with silk sutures.

LPS, M-CSF and SDF-1α-injected mice were sacrificed 1 week after the injection and the brains were processed for immunohistochemistry as described above. The following antibodies were used: A13 pan antibody (Biosource, Belgium) to detect human Aβ, CD11b and I-A/I-E antibody recognizing MHC class II alloantigens (Serotec, UK) to detect microglia using TSA amplification system (PerkinElmer, Boston, MA, USA) according to the manufacturer's instructions. Immunoreactive cells were counted in 4-6 hippocampal sections per mouse from an area containing the hippocampal subfields stratum pyramidale, radiatum, laconosum-moleculare and molecular layers of the dentate gyrus.

To assess the effect of LPS, M-CSF and SDF-1α injection on A13 burden, the sections were imaged with an Olympus AX70 microscope (Olympus, N.Y., USA) with an attached digital camera (Color View 12, Soft Imaging System, Munster, Germany) running AnalySIS software (Soft Imaging System, Munster, Germany). Aβ pan immunoreactive area in the hippocampi was quantified from 4-5 representative sections per animal using ImagePro Plus software (Media Cybernetics, Silver Spring, Md., USA). Data are expressed as the percent area of the hippocampi occupied by immunoreactivity (Aβ burden) and presented as mean±SEM.

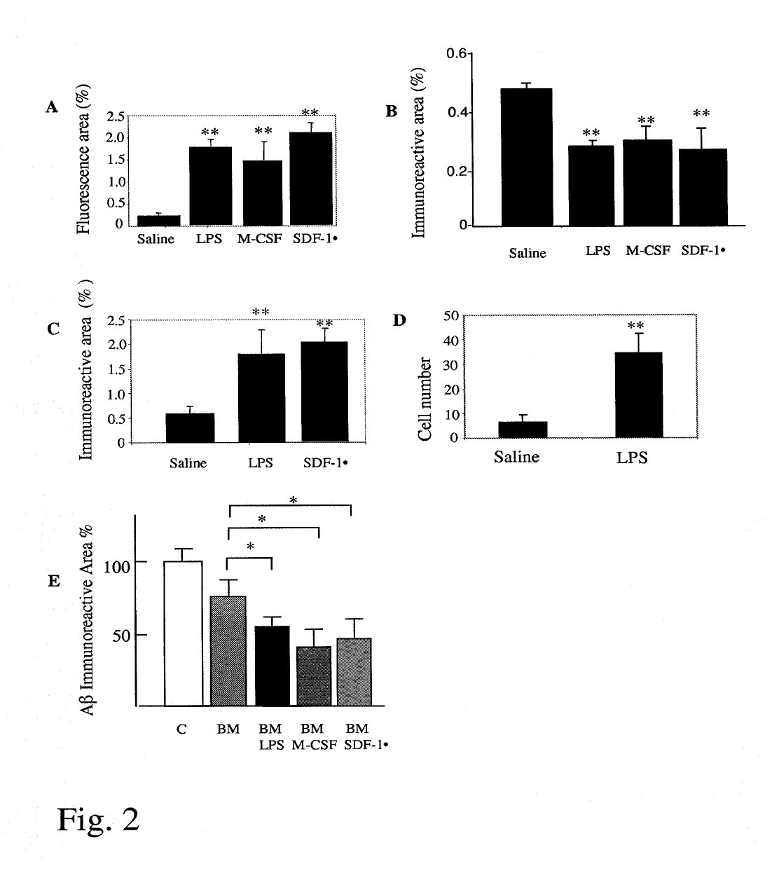

It was observed that LPS, M-CSF and SDF-1α injection increased the number of BM-derived cells about 10-fold (FIG. 2A) and reduced amyloid burden by over 40% (repeated measures ANOVA, p<0.05) (FIG. 2B). BM-derived cells were in activated state after LPS, M-CSF and SDF-1α injections as judged by the immunoreactivity for MHCII, a marker of phagocytotic cells. On the other hand, MHCII positive cells were eGFP positive, indicating that a vast majority of activated cells were BM-derived (FIG. 2C). After LPS, M-CSF and SDF-1α injection BM-derived cells contained amyloid or were closely associated with beta-amyloid, indicating that LPS, M-CSF and SDF-1α induced ability of BM-derived cells to phagocyte amyloid (FIG. 2D).

### Example 3

### LPS, M-CSF and SDF-1α Enhanced Beta-Amyloid Degradation (Clearance) by BM Cells In Vitro

BM cells from adult eGFP transgenic mice were cultured as described (Servet-Delprat et al., BMC Immunol. 3:15 (2002). Aged double transgenic APP+PS1 (see (2)) were perfused with saline and the brains containing human beta-amyloid deposits were frozen on dry ice. Sagittal sections (10 μm) were cut on a cryostat (Leica), mounted on poly-L-lysine-coated coverslips, transferred to two-well chamber slides and used immediately or stored at −80° C. until use. BM-derived eGFP expressing cells were seeded in the chamber at a density of 5×10⁶ cells in 1 ml of assay medium (DMEM/F12, G5 supplement, 0.2% bovine serum albumin (BSA), penicillin and streptomycin) and the cultures were maintained for 24 h or longer at 37° C. (Wyss-Coray et al., Nat Med. 9, 453-457 (2003)). The amyloid burden was analyzed using immunohistochemistry and image analysis. Beta-amyloid was detected using a pan-Aβ antibody. The percent area of the mouse hippocampus occupied by fluorescent Aβ staining (Aβ amyloid burden, respectively) was measured in at least 8 sections per treatment (control, 10 ng/ml LPS, 500 U/ml M-CSF, 500 ng/ml SDF-1α). The sections were imaged with a Nikon microscope attached to a ColorView digital camera, and Image-Pro Plus software was used for automated counting of the number of pixels above the threshold and computing the Aβ burden in the hippocampal area.

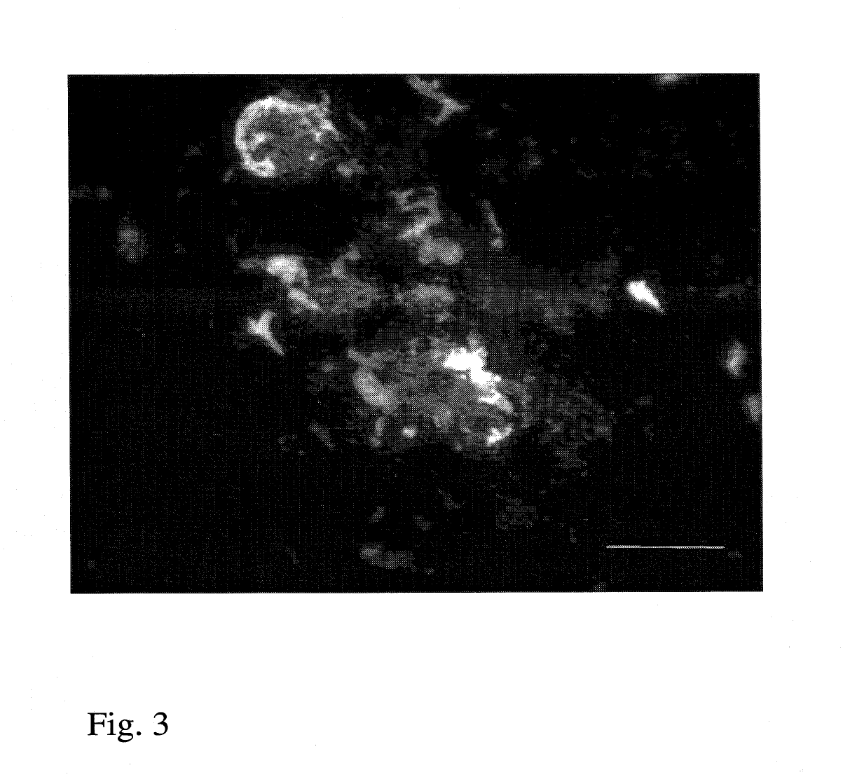

It was observed that LPS, M-CSF and SDF-1α stimulate phagocytotic properties of BM cells and that these stimulated cells were able to clear or degrade beta-amyloid. BM cells alone were able to clear beta-amyloid, but the clearance was more than doubled after treatment with LPS, M-CSF or SDF-1α (FIG. 2 E, *repeated measures ANOVA, p<0.05). Confocal microscopy demonstrated that the stimulated cells are closely associated with beta-amyloid, suggesting phagocytosis (FIG. 3).

This invention has been described with an emphasis upon some of the preferred embodiments and applications. However, it will be apparent to those skilled in the art that variations in the disclosed embodiments can be prepared and used and that the invention can be practiced otherwise than as specifically described herein within the scope of the following claims.

1-18. (canceled)

19. A method of treating a subject suffering from an amyloid-accumulating disorder, comprising:
administering to the subject an effective amount of a pharmaceutical composition comprising a phagocytotic bone marrow cell.

20. The method according to claim 19, wherein the pharmaceutical composition further comprises a phagocytosis-stimulating agent selected from the group consisting of a lipopolysaccharide (LPS), a macrophage colony stimulating factor (M-CSF) and a stromal cell derived factor 1-alpha (SDF 1-α) or combinations thereof.

21. The method according to claim 19, wherein the pharmaceutical composition further comprises a pharmaceutically acceptable carrier.

22. The method according to claim 19, wherein the amyloid-accumulating disorder is selected from the group consisting of Alzheimer's disease, Parkinson's disease, Huntington's disease, amyotrophic lateral sclerosis (ALS), transmissible spongiform encephalopathy, type II diabetes and familial and secondary amyloid diseases (amyloidosis).

23. The method according to claim 19, wherein the phagocytotic bone marrow cell is a mammalian bone marrow cell.

24. The method according to claim 23, wherein the phagocytotic bone marrow cell is a human bone marrow cell.

25. The method according to claim 23, wherein the phagocytotic bone marrow cell is a mouse bone marrow cell.

26. The method according to claim 23, wherein the phagocytotic bone marrow cell is a rat bone marrow cell.

27. The method according to claim 20, wherein the phagocytosis-stimulating agent is LPS.

28. The method according to claim 20, wherein the phagocytosis-stimulating agent is M-CSF.

29. The method according to claim 20, wherein the phagocytosis-stimulating agent is SDF 1-α.

30. The method according to claim 19, wherein administering comprises parenteral administration.

31. A method of degrading beta-amyloid peptides or aggregates thereof in tissue of a subject, comprising:
administering to the subject an effective amount of a pharmaceutical composition comprising a phagocytotic bone marrow cell.

32. The method of claim 31, wherein the amyloid aggregates comprise a protein selected from the group consisting of beta-amyloid, tau protein, proteinase K resistant isoform (PrP-res) of prion protein, islet amyloid polypeptide (IAPP), parkin, alpha-synuclein, synphilin and SOD1.

33. The method according to claim 32, wherein the protein is beta-amyloid.
